(19) United States
(12) Patent Application Publication
(10) Pub. No.: US 2023/0405271 A1
(43) Pub. Date: Dec. 21, 2023

(54) CATHETER AND METHOD FOR MANUFACTURING SAME

(71) Applicant: TERUMO KABUSHIKI KAISHA, Tokyo (JP)

(72) Inventor: Kenta SUZUKI, Fujinomiya (JP)

(21) Appl. No.: 18/237,756

(22) Filed: Aug. 24, 2023

Related U.S. Application Data
(63) Continuation of application No. PCT/JP2022/003427, filed on Jan. 28, 2022.

(30) Foreign Application Priority Data
Mar. 9, 2021 (JP) 2021-037684

(51) Int. Cl. A61M 25/00

(52) U.S. Cl. CPC A61M 25/0074; A61M 25/0045; A61M 25/001

(57) ABSTRACT

A catheter includes a cylindrical distal member connected to a distal end part of a catheter body, and the distal member has a bending portion located distal to a most distal end part of the catheter body, the bending portion being a section that bends when an external force in a bending direction is applied to a distal end part of the distal member with the distal end part of the catheter body being fixed. The Young's modulus of the bending portion is smaller than the Young's modulus of the proximal end part of the distal member. A method for manufacturing the catheter includes performing a heat treatment for fusing the proximal end part of the distal member to the catheter body while adjusting a thermal load on an axially intermediate part of the distal member to be smaller than a thermal load on the proximal end part.

### CROSS-REFERENCE TO RELATED APPLICATIONS

The present disclosure is a continuation of and claims benefit to PCT/JP2022/003427 filed on Jan. 28, 2022, entitled “CATHETER AND METHOD FOR MANUFACTURING SAME” which claims priority to Japanese Patent Application No. 2021-037684 filed on Mar. 9, 2021. The entire disclosure of the applications listed above are hereby incorporated herein by reference, in their entireties, for all that they teach and for all purposes.

### BACKGROUND

The present disclosure relates to a catheter and a method for manufacturing the same.

A catheter such as a balloon catheter, a microcatheter, or a guiding catheter has a cylindrical distal member that is connected to a distal end part of a catheter body (e.g., in the case of a balloon catheter, at least one of an inner tube of a shaft and a balloon) and allows a guide wire to pass therethrough (see, for example, Japanese Patent Publication No. 2011-56148 A). The distal member is more flexible than the distal end part of the catheter body, and has trackability so that the distal member can be deformed following the shape of the guide wire in order to reach the target position without being caught by an obstacle along the guide wire in the body cavity.

### BRIEF SUMMARY

A conventional catheter has a configuration in which a bending portion of a distal member is located at the most distal end part of a catheter body, the bending portion being a section that bends when an external force in a bending direction is applied to a distal end part of the distal member with the distal end part of the catheter body being fixed. Therefore, there is room for improvement in the trackability of the distal member with respect to the guide wire.

At least one object of the present disclosure is to provide a catheter capable of achieving high trackability of a distal member with respect to a guide wire, and a method for manufacturing the catheter.

A catheter according to at least one aspect of the present disclosure includes a cylindrical distal member connected to a distal end part of a catheter body, and the distal member has a bending portion located distal to a most distal end part of the catheter body, the bending portion being a section that bends when an external force in a bending direction is applied to a distal end part of the distal member with the distal end part of the catheter body being fixed.

As an embodiment of the present disclosure, in the catheter, the distal end part of the distal member is tapered.

As an embodiment of the present disclosure, in the catheter, the distal end part of the distal member is formed of a thermoplastic resin.

As an embodiment of the present disclosure, in the catheter, the distal end part of the distal member includes only at least one thermoplastic resin layer.

As an embodiment of the present disclosure, in the catheter, the Young's modulus of the bending portion is smaller than the Young's modulus of a proximal end part of the distal member.

A catheter according to a second aspect of the present disclosure includes a distal member having a cylindrical shape and connected to a distal end part of a catheter body, wherein the distal member has a bending portion having a Young's modulus smaller than a Young's modulus of a proximal end part of the distal member, the bending portion being a section that bends when an external force in a bending direction is applied to a distal end part of the distal member with the distal end part of the catheter body being fixed.

As an embodiment of the present disclosure, in the catheter, the Young's modulus of the bending portion of the distal member is smaller than the Young's modulus of the distal end part of the distal member.

A method for manufacturing a catheter according to a third aspect of the present disclosure includes a heat treatment step of performing a heat treatment for fusing a proximal end part of a distal member having a cylindrical shape to a catheter body while adjusting a thermal load on an axially intermediate part of the distal member to be smaller than a thermal load on the proximal end part.

As an embodiment of the present disclosure, in the method for manufacturing a catheter, the heat treatment step includes a heat transfer step of transferring heat to the proximal end part through a heat transfer portion that has a cylindrical shape and that contracts by heat.

As an embodiment of the present disclosure, in the method for manufacturing a catheter, the heat transfer portion absorbs radiation and generates heat.

As an embodiment of the present disclosure, in the method for manufacturing a catheter, the heat treatment step includes a distal end treatment step of applying a thermal load larger than a thermal load on the axially intermediate part of the distal member to the distal end part of the distal member.

The present disclosure can provide a catheter capable of achieving high trackability of a distal member with respect to a guide wire, and a method for manufacturing the catheter.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages are described herein and will be apparent to those skilled in the art upon consideration of the following Detailed Description and in view of the figures.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is an external view illustrating a catheter in accordance with embodiments of the present disclosure;

FIG. 2 is a vertical cross-sectional view illustrating a distal end part of the catheter illustrated in FIG. 1;

FIG. 3 is a partially enlarged view of FIG. 2;

FIG. 4 is a schematic diagram illustrating an example of a bending test for confirming a position of a bending portion of a distal member of the catheter illustrated in FIG. 1;

FIG. 5 is a schematic diagram illustrating a state where the bending portion bends by the bending test illustrated in FIG. 4;

FIG. 6 is a schematic diagram illustrating a state where a bending portion of a distal member of a catheter as a comparative example bends by the bending test illustrated in FIG. 4;

FIG. 7 is a schematic diagram illustrating a member for heat treatment used for manufacturing the catheter illustrated in FIG. 1;

FIG. 8 is a partially enlarged view of a catheter in accordance with embodiments of the present disclosure;

FIG. 9 is a schematic diagram illustrating a state where a bending portion of the catheter illustrated in FIG. 8 bends by the bending test illustrated in FIG. 4;

FIG. 10 is a schematic diagram illustrating a state where a bending portion of a distal member of a catheter as a comparative example bends by the bending test illustrated in FIG. 4;

FIG. 11 is a partially enlarged view of a catheter in accordance with embodiments of the present disclosure;

FIG. 12 is a partially enlarged view of a catheter in accordance with embodiments of the present disclosure; and

FIG. 13 is a partially enlarged view of a catheter in accordance with embodiments of the present disclosure.

### DETAILED DESCRIPTION

Hereinafter, embodiments of a catheter and a method for manufacturing the same according to the present disclosure will be described in detail by way of examples with reference to the drawings.

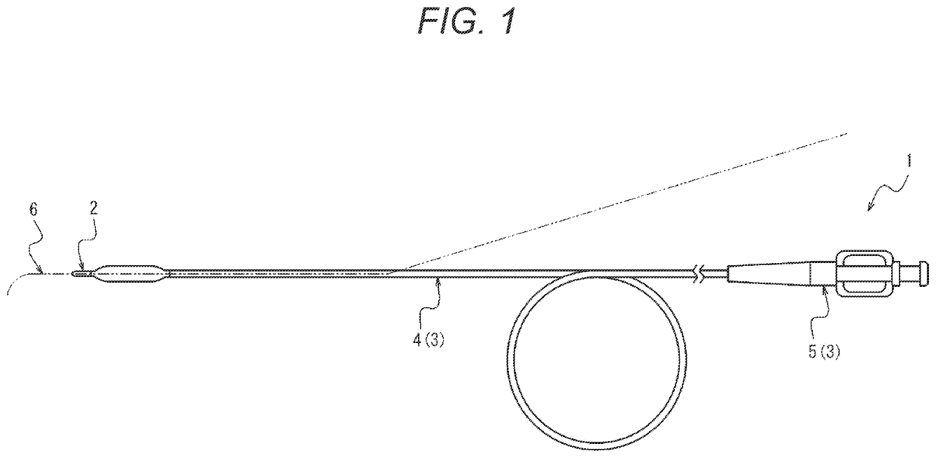

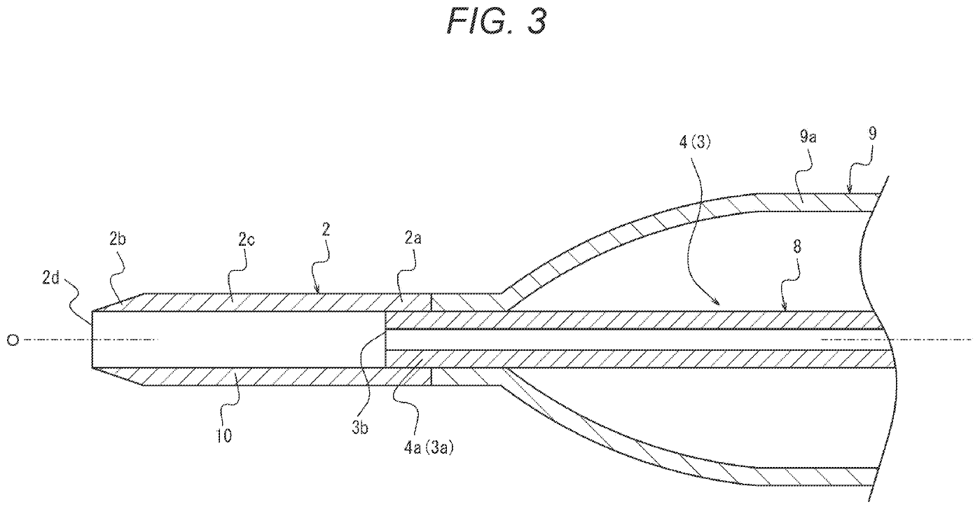

As illustrated in FIGS. 1 to 3, a catheter 1 according to at least one embodiment of the present disclosure includes a cylindrical distal member 2 extending along a central axis O, and a catheter body 3 including a distal end part 3a connected to a proximal end part 2a of the distal member 2. The catheter body 3 includes a shaft portion 4 having a distal end part 4a connected to the proximal end part 2a of the distal member 2 and having an elongated shape coaxial with the distal member 2, and a hub 5 having a distal end part connected to a proximal end part of the shaft portion 4. The distal member 2 and the shaft portion 4 have flexibility (e.g., bendability), so that they can enter a lumen such as a vessel (e.g., a blood vessel, etc.) in a living body (e.g., a human body, an animal body, etc.), that is, a body cavity, along a curved guide wire 6.

In the present disclosure, a direction along the central axis O of the distal member 2 is referred to as an axial direction, a direction along a straight line orthogonal to the central axis O is referred to as a radial direction, a direction around the central axis O is referred to as a circumferential direction, a cross section including the central axis O is referred to as a longitudinal cross section, an end on a side inserted into a body cavity during an operation is referred to as a distal end part, and an end on an opposite side, that is, on a side closer to a practitioner is referred to as a proximal end part.

The shaft portion 4 includes an outer tube 7, an inner tube 8, and a balloon 9. That is, the catheter 1 is a balloon catheter. However, the catheter 1 is not limited to a balloon catheter, and may be some other type of catheter, for example, a microcatheter or a guiding catheter.

The outer tube 7 has an elongated cylindrical shape extending in the axial direction. The proximal end part of the outer tube 7 is connected to the distal end part of the hub 5. The distal end part of the outer tube 7 is connected to the proximal end part of the balloon 9.

The balloon 9 has a cylindrical shape in which the distal end part and the proximal end part extend in the axial direction, and an axially intermediate part between the distal end part and the proximal end part constitutes a cylindrical balloon body 9a that is capable of being enlarged in the radial direction. FIGS. 1 to 3 illustrate the deployed balloon body 9a expanded in the radial direction. Before being deployed, the balloon body 9a is in an undeployed state where the balloon body 9a is folded to have the same outer diameter as the outer tube 7. The distal end part of the balloon 9 is connected to the distal end part of the inner tube 8. More specifically, the inner peripheral surface of the balloon 9 at the distal end part is joined to the outer peripheral surface of the inner tube 8 at the distal end part by, for example, fusion (e.g., fusion bonding, welding, etc.).

The inner tube 8 has a long cylindrical shape. The most distal end part of the inner tube 8 is positioned distal to the most distal end part of the balloon 9. The distal end part and the axially intermediate part of the inner tube 8 extend in the axial direction. The proximal end part of the inner tube 8 extends obliquely outward in the radial direction toward the proximal side. The most proximal end part of the inner tube 8 is joined to an outer peripheral edge of an oval notch provided in the peripheral surface of the outer tube 7 so as to be in close contact with the outer peripheral edge over the entire circumference.

A communication path communicating with the lumen of the balloon body 9a is formed between the outer tube 7 and the inner tube 8. A fluid is sent to the lumen of the balloon body 9a through the communication path, by which the balloon body 9a in the undeployed state can be transitioned to the deployed state.

The proximal end part 2a of the distal member 2 is joined to the distal end part 4a of the shaft portion 4 of the catheter body 3 by fusion. More specifically, the inner peripheral surface of the distal member 2 at the proximal end part 2a is joined to the outer peripheral surface of the inner tube 8 at the distal end part by fusion, and the proximal end of the proximal end part 2a of the distal member 2 is joined to the distal end of the distal end part of the balloon 9 by fusion.

During operation, the guide wire 6 passes through the distal member 2 and the lumen of the inner tube 8. The catheter 1 is of a rapid exchange (RX) catheter type in which the proximal end part of the lumen through which the guide wire 6 passes is positioned at an axially intermediate part of the shaft portion 4. However, the catheter 1 is not limited to the RX type, and may be, for example, of an over-the-wire (OTW) catheter type.

Each of the outer tube 7, the inner tube 8, and the balloon 9 can be made of, for example, polyolefins (for example, polyethylene, polypropylene, polybutene, ethylene-propylene copolymer, ethylene-vinyl acetate copolymer, ionomer, etc., or a mixture of two or more kinds thereof), a polymer material such as polyvinyl chloride, polyamide, polyamide elastomer, polyurethane, polyurethane elastomer, polyimide, or fluororesin, or a mixture of two or more kinds of the polymer materials.

Each of the outer tube 7, the inner tube 8, and the balloon 9 may have a single-layer structure or a multi-layer structure. Each of the outer tube 7, the inner tube 8, and the balloon 9 may have a structure in which the same type of material is connected over the entire length in the axial direction, or a structure in which different types of materials are connected in the axial direction.

A fused portion of the shaft portion 4 fused to at least the distal member 2 and the distal member 2 are formed of, for example, a thermoplastic resin such as a polyamide resin or a polyolefin resin. The distal member 2 may include only one thermoplastic resin layer. However, the distal member 2 is not limited thereto, and may include only two or more thermoplastic resin layers, or may have a layer other than the thermoplastic resin layer.

The distal member 2 may have, for example, an inner diameter of 0.42 mm and an outer diameter of 0.56 mm. The distal member 2 can be formed of, for example, a polyamide elastomer (e.g., Grilamid® ELG 5660 manufactured by EMS).

The distal end part 2b of the distal member 2 is tapered. More specifically, the outer peripheral surface of the distal end part 2b of the distal member 2 is a linear inclined surface inclined radially inward toward the distal side in the longitudinal cross section. However, the outer peripheral surface of the tapered distal end part 2b of the distal member 2 is not limited thereto, and may be, for example, a rounded surface having a curved shape such as an arc shape in the longitudinal cross section. The distal end part 2b of the distal member 2 is not limited to having a tapered shape.

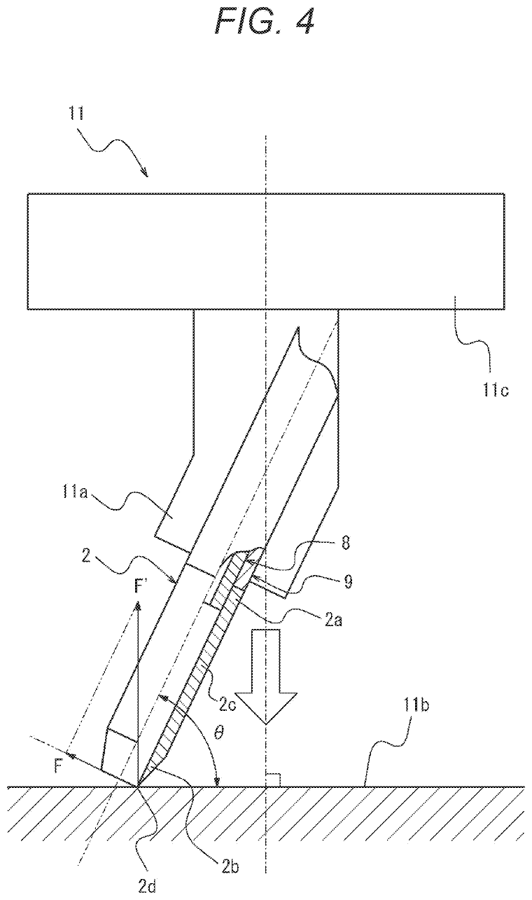

An axially intermediate part 2c, which is a section connecting the distal end part 2b and the proximal end part 2a in the distal member 2, has a bending portion 10 which bends (in other words, is curved) when an external force F (see, e.g., FIG. 4) in the bending direction is applied to the distal end part 2b of the distal member 2 with the distal end part 3a of the catheter body 3 being fixed. In this manner, the bending portion 10 of the distal member 2 is positioned distal to the most distal end part 3b of the catheter body 3 (that is, the most distal end part of the inner tube 8).

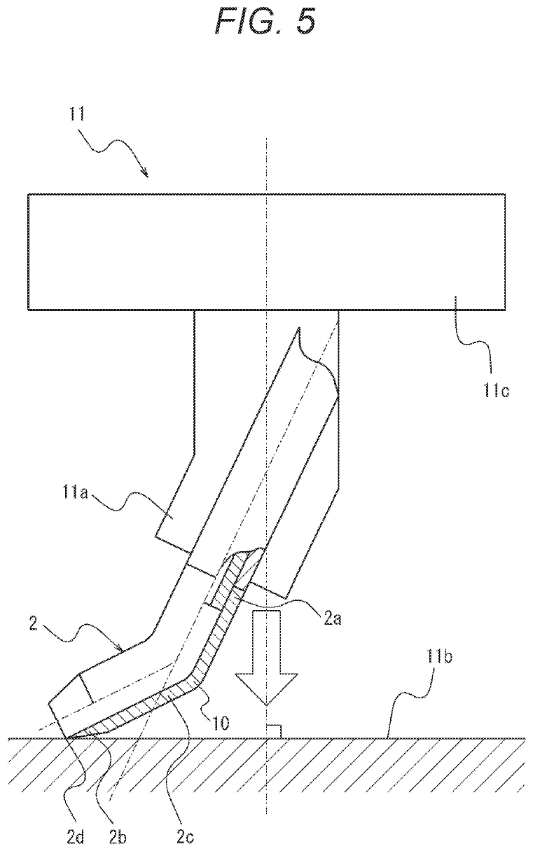

The position of the bending portion 10 can be confirmed, for example, by a bending test illustrated in FIGS. 4 and 5 as an example. The bending test in this example uses a testing device 11 provided with a grip portion 11a (e.g., chuck, collet, clamp, gripper, etc.) that grips the distal end part 3a of the catheter body 3, a contact surface 11b with which the most distal end part 2d of the distal member 2 comes in contact, and a drive unit 11c that relatively moves the grip portion 11a toward the contact surface 11b at a predetermined speed. According to the testing device 11 described above, it is possible to fix the distal end part 3a of the catheter body 3 and apply the external force F in the bending direction to the distal end part 2b of the distal member 2. The external force F is a radially inner component of the reaction force F′ from the contact surface 11b. The testing device 11 is configured as, for example, a micro autograph in which the grip portion 11a is configured by a chuck and the drive unit 11c is configured by a load cell. The contact surface 11b is formed of, for example, silicone rubber.

As a test method, for example, an angle θ of inclination of the central axis O of the distal member 2 with respect to the contact surface 11b is set in a range of 60° to 80° (plus or minus 5°), and the grip portion 11a is moved toward the contact surface 11b at a relative speed of 2 mm/min as illustrated in FIG. 4 (e.g., in a direction normal to the contact surface 11b).

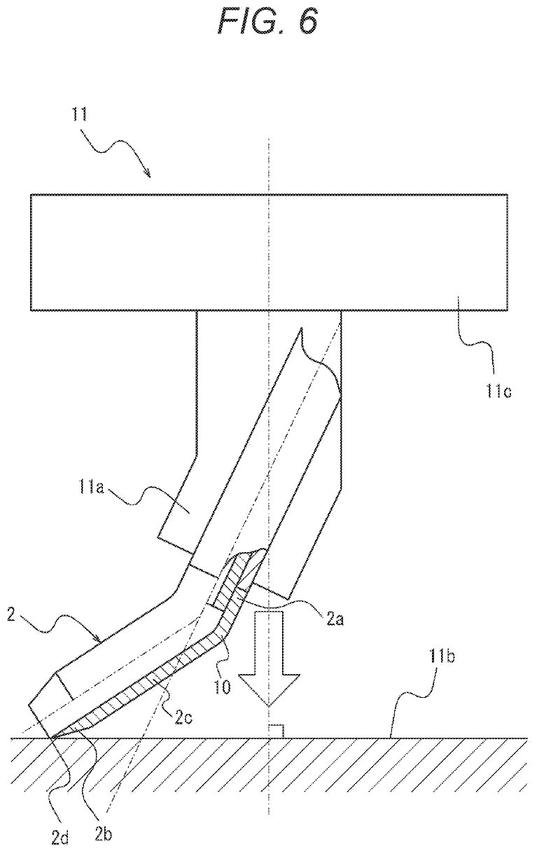

The section that bends, as illustrated in FIG. 5 by the test described above, is the bending portion 10. Since the Young's modulus (e.g., the modulus of elasticity in tension or compression, etc.) of the bending portion 10 is smaller than the Young's modulus of the proximal end part 2a of the distal member 2, the bending portion 10 is positioned distal to the most distal end part 3b of the catheter body 3. In contrast to the present embodiment, in a comparative example in which the Young's modulus of the distal member 2 is constant over the entire length in the axial direction, the bending portion 10 is located at the most distal end part 3b of the catheter body 3 as illustrated in FIG. 6. Alternatively, the distal member 2 in the comparative example may buckle in a bellows shape over the entire length in the axial direction without forming the bending portion 10.

The bending portion 10 of the present embodiment can be formed through a heat treatment step of performing a heat treatment for fusing the proximal end part 2a of the distal member 2 to the catheter body 3 while adjusting a thermal load on the axially intermediate part 2c of the distal member 2 to be smaller than a thermal load on the proximal end part 2a of the distal member 2. That is, the method for manufacturing the catheter according to the present embodiment includes the heat treatment step described above.

The heat treatment step includes a distal end treatment step of applying a thermal load larger than the thermal load to the axially intermediate part 2c of the distal member 2 to the distal end part 2b of the distal member 2. The distal end part 2b of the distal member 2 is formed in a tapered shape by the distal end treatment step.

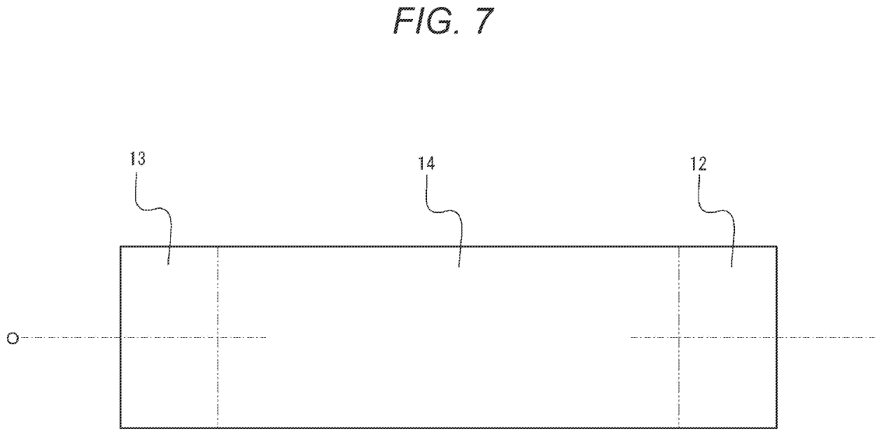

The heat treatment step also includes a heat transfer step of transferring heat to the proximal end part 2a of the distal member 2 via a cylindrical first heat transfer portion 12 that contracts by heat and transferring heat to the distal end part 2b of the distal member 2 via a cylindrical second heat transfer portion 13 that contracts by heat (see, e.g., FIG. 7). Due to the heat transfer step described above, a thermal load larger than the thermal load to the axially intermediate part 2c of the distal member 2 can be applied to the proximal end part 2a and the distal end part 2b of the distal member 2. In the heat treatment step, core bars corresponding to the lumen of the distal member 2 and the lumen of the catheter body 3 are inserted in advance. In the present embodiment, core bars having outer diameters respectively corresponding to the diameter of the lumen of the distal member 2 and the lumen of the catheter body 3 are used.

Due to the suppression of the thermal load on the axially intermediate part 2c of the distal member 2, it is possible to prevent a situation in which a thermoplastic resin constituting the axially intermediate part 2c of the distal member 2 is melted and cured again to cause a change in composition, so that the thermoplastic resin is harder than before being melted, that is, to prevent a decrease in flexibility (that is, an increase in Young's modulus) due to the thermal load on the axially intermediate part 2c of the distal member 2.

The first heat transfer portion 12 absorbs radiation to generate heat, thereby contracting, and accordingly comes into contact with the outer peripheral surface of the distal member 2 at the proximal end part 2a and transfers heat. The second heat transfer portion 13 absorbs radiation to generate heat, thereby contracting, and accordingly comes into contact with the outer peripheral surface of the distal member 2 at the distal end part 2b and transfers heat.

In some embodiments, the transfer of heat from the first heat transfer portion 12 to the outer peripheral surface of the distal member 2 at the proximal end part 2a may cause a change in material composition or structure to the proximal end part 2a of the distal member 2. For example, the material at the proximal end part 2a of the distal member 2 may melt and then cure such that the proximal end part 2a of the distal member 2 transforms into a harder section of the distal member 2 than the bending portion 10 of the distal member 2 (e.g., the Young's modulus of the proximal end part 2a of the distal member 2 is transformed to be greater than the Young's modulus of the bending portion 10 of the distal member 2). Additionally or alternatively, the transfer of heat from the second heat transfer portion 13 to the outer peripheral surface of the distal member 2 at the distal end part 2b may cause a change in material composition or structure to the distal end part 2b of the distal member 2. For instance, the material at the distal end part 2b of the distal member 2 may melt and then cure such that the distal end part 2b of the distal member 2 transforms into a harder section of the distal member 2 than the bending portion 10 of the distal member 2 (e.g., the Young's modulus of the distal end part 2b of the distal member 2 is transformed to be greater than the Young's modulus of the bending portion 10 of the distal member 2). In some embodiments, the proximal end part 2a of the distal member 2 and the distal end part 2b of the distal member 2 may be heated (e.g., heat-transformed, melted-and-cured, etc.) to have the same, or equal, Young's modulus, which is greater than the Young's modulus of the bending portion 10 of the distal member 2.

In some embodiments, the distal member 2 may be made from a single unitary, undivided, or continuous, piece of material that is capable of being heat-transformed into having different material properties (e.g., different Young's moduli) at two or more sections thereof (e.g., the proximal end part 2a of the distal member 2, the distal end part 2b of the distal member 2, and/or the bending portion 10). At least some benefits to this arrangement includes providing a catheter 1 having a seamless, unified, and low cost distal member 2 that is capable of flexing only along a controlled area (e.g., the bending portion 10) without requiring complex machining, separately fused component sections, and/or other high-friction surfaces or edges.

Each of the first heat transfer portion 12 and the second heat transfer portion 13 is configured by, for example, a colored tube colored in black or the like that easily absorbs energy applied by a laser as radiation. The first heat transfer portion 12 and the second heat transfer portion 13 are connected by means of, for example, a connection portion 14 configured by a transparent tube that hardly absorbs (or is incapable of absorbing) the energy provided by a laser. Alternatively, the first heat transfer portion 12 and the second heat transfer portion 13 are disposed separately from each other. In any event, as the laser energy is applied to the first heat transfer portion 12 and the second heat transfer portion 13 of the tubular heat treatment member, the first heat transfer portion 12 contracts and contacts the outer periphery of the proximal end part 2a of the distal member 2 and the second heat transfer portion 13 contracts and contacts the outer periphery of the distal end part 2b of the distal member 2. The contacting portions (e.g., the first heat transfer portion 12 and the second heat transfer portion 13) transfer heat energy to the proximal end part 2a of the distal member 2 and the distal end part 2b of the distal member 2, respectively. This heat energy may cause the proximal end part 2a and the distal end part 2b of the distal member 2 to melt and then cure at a higher or greater Young's modulus than the Young's modulus of the bending portion 10 of the distal member 2.

The distal member 2 can be formed by common extrusion including coating. However, the method for manufacturing the distal member 2 is not limited thereto. The method for manufacturing the inner tube 8 and the like is also not particularly limited.

In the catheter 1 according to the present embodiment described above, the bending portion 10 of the distal member 2 is located distal to the most distal end part 3b of the catheter body 3, whereby the distal member 2 is easily flexibly deformed into a shape along the curved guide wire 6 at the time of operation, and high trackability of the distal member 2 to the guide wire 6 can be achieved.

In addition, in the catheter 1 according to the present embodiment, the distal end part 2b of the distal member 2 is tapered, whereby the crossability for advancing in the body cavity along the guide wire 6 at the time of operation can be enhanced, and the occurrence of curling in which the distal end part 2b of the distal member 2 is deformed to be curled can be suppressed.

In the catheter 1 according to the present embodiment, the distal member 2 is formed of a thermoplastic resin, whereby the distal member 2 can be easily joined to the catheter body 3 by fusion.

In the catheter 1 according to the present embodiment, the distal member 2 includes only at least one thermoplastic resin layer, whereby the bending portion 10 of the distal member 2 can be easily formed by performing a heat treatment for fusing the distal member 2 to the catheter body 3 while adjusting a thermal load on the axially intermediate part 2c of the distal member 2 to be smaller than a thermal load on the proximal end part 2a of the distal member 2.

In the catheter 1 according to the present embodiment, the Young's modulus of the bending portion 10 is smaller than the Young's modulus of the proximal end part 2a of the distal member 2, whereby the bending portion 10 can be formed with a simple structure.

In addition, in the method for manufacturing a catheter according to the present embodiment, the bending portion 10 located distal to the most distal end part 3b of the catheter body 3 can be formed in the distal member 2 by the heat treatment step, whereby high trackability of the distal member 2 with respect to the guide wire 6 can be achieved.

In the method for manufacturing the catheter according to the present embodiment, the heat treatment step includes the heat transfer step, whereby the heat treatment step can be easily performed.

In the method for manufacturing the catheter according to the present embodiment, the first heat transfer portion 12 and the second heat transfer portion 13 each absorb radiation and generate heat, whereby the heat transfer step can be easily performed.

In the method for manufacturing the catheter according to the present embodiment, the heat treatment step includes the distal end treatment step, whereby the distal end part 2b of the distal member 2 can be easily formed into a tapered shape.

The above-described embodiment is provided as an example of the present disclosure, and various modifications as described below, for example, are possible.

For example, various changes are possible for the catheter 1, as long as the catheter 1 has the cylindrical distal member 2 connected to the distal end part 3a of the catheter body 3, and the bending portion 10 of the distal member 2 is located distal to the most distal end part 3b of the catheter body 3, the bending portion being a section that bends when the external force F in the bending direction is applied to the distal end part 2b of the distal member 2 with the distal end part 3a of the catheter body 3 being fixed.

However, the distal end part 2b of the distal member 2 may be tapered.

In addition, the distal member 2 may be formed of a thermoplastic resin.

The distal member 2 may include only one thermoplastic resin layer.

The Young's modulus of the bending portion 10 may be smaller than the Young's modulus of the proximal end part 2a of the distal member 2.

In addition, the inner peripheral surface of the balloon 9 at the distal end part may be joined to the outer peripheral surface of the distal member 2 at the proximal end part 2a by, for example, fusion in place of or in addition to the outer peripheral surface of the inner tube 8 at the distal end part.

Alternatively, various changes are possible for the catheter 1, as long as the catheter 1 has the cylindrical distal member 2 connected to the distal end part 3a of the catheter body 3, and the Young's modulus of the bending portion 10 of the distal member 2 is smaller than the Young's modulus of the proximal end part 2a of the distal member 2, the bending portion being a section that bends when the external force F in the bending direction is applied to the distal end part 2b of the distal member 2 with the distal end part 3a of the catheter body 3 being fixed.

Various changes are possible for the method for manufacturing a catheter as long as the method includes a heat treatment step of performing a heat treatment for fusing the proximal end part 2a of the distal member 2 to the catheter body 3 while adjusting a thermal load on the axially intermediate part 2c of the distal member 2 to be smaller than a thermal load on the proximal end part 2a of the distal member 2.

However, the heat treatment step may include a heat transfer step of transferring heat to the proximal end part 2a of the distal member 2 through the cylindrical first heat transfer portion 12 that contracts by heat.

In addition, the first heat transfer portion 12 may absorb radiation to generate heat.

The heat treatment step may include a distal end treatment step of applying a thermal load larger than the thermal load to the axially intermediate part 2c of the distal member 2 to the distal end part 2b of the distal member 2.

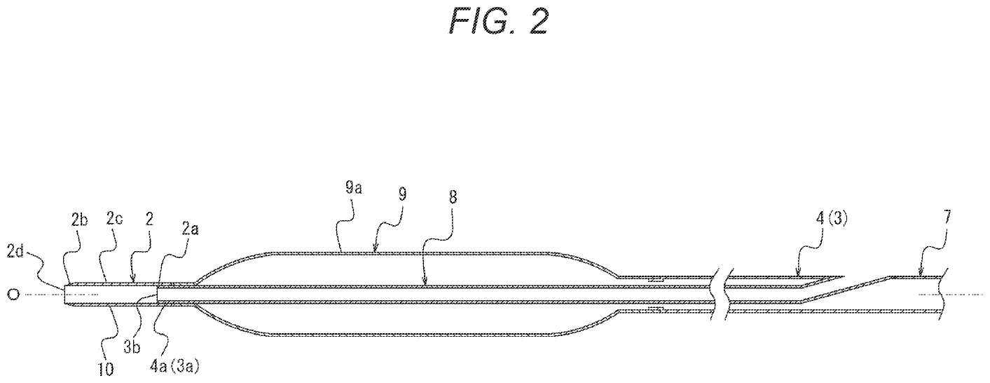

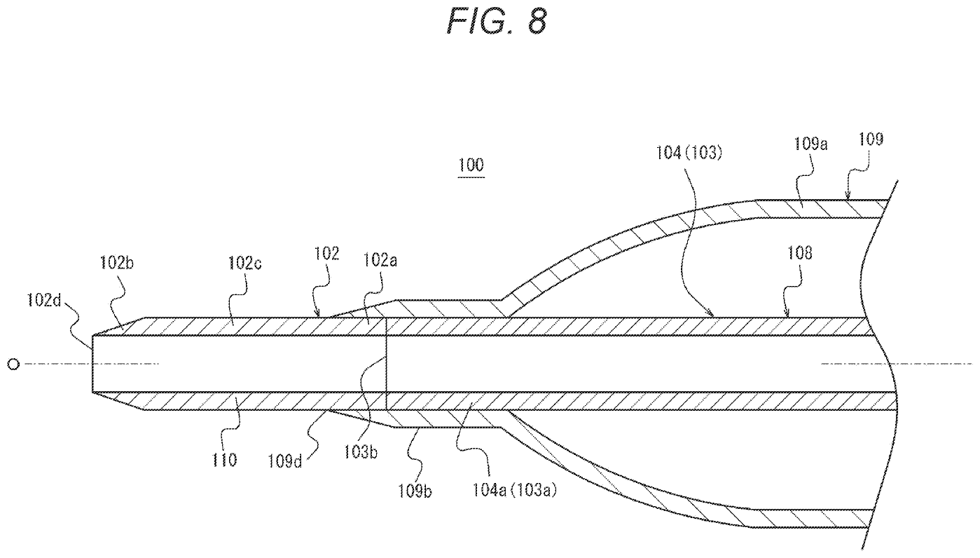

Next, a catheter 100 according to another embodiment of the present disclosure will be described with reference to FIG. 8, that is a partially enlarged view of the catheter 100. Note that the components in FIG. 8 that are common to FIGS. 1 and 2 are denoted by the same reference numerals as those in FIGS. 1 and 2, and the details thereof are assumed to be the same and are omitted.

As illustrated in FIG. 8, the catheter 100 according to another embodiment includes a cylindrical distal member 102 extending along a central axis O, and a catheter body 103 including a distal end part 103a connected to a proximal end part 102a of the distal member 102. The catheter body 103 includes a shaft portion 104 having the distal end part 103a connected to the proximal end part 102a of the distal member 102 and having an elongated shape coaxial with the distal member 102, and a hub 5 having a distal end part connected to a proximal end part of the shaft portion 104. The distal member 102 and the shaft portion 104 have flexibility (e.g., bendability, etc.), so that they can enter a lumen such as a vessel (e.g., a blood vessel, etc.) in a living body (e.g., a human body), that is, a body cavity, along a curved guide wire 6.

The balloon 109 has a cylindrical shape in which a distal end part 109b and a proximal end part extend in the axial direction, and an axially intermediate part between the distal end part and the proximal end part constitutes a cylindrical balloon body 109a enlarged in the radial direction. FIG. 8 illustrates the deployed balloon body 109a expanded in the radial direction. Before being deployed, the balloon body 109a is in an undeployed state where the balloon body 109a is folded to have the same outer diameter as the outer tube 7. The distal end part 109b of the balloon 109 is disposed across the distal end part of the inner tube 108 (e.g., the distal end part 103a of the catheter body 103) and the proximal end part 102a of the distal member 102. More specifically, the inner peripheral surface of the balloon 109 at the distal end part 109b is joined to the outer peripheral surface of the catheter body 103 at the distal end part 103a by, for example, fusion.

The inner tube 108 has a long cylindrical shape. The most distal end part of the inner tube 108, that is, the most distal end part 103b of the catheter body 103, is positioned proximal to the most distal end part 109d of the balloon 109.

The proximal end part 102a of the distal member 102 is joined to the distal end part 104a of the shaft portion 104 of the catheter body 103 by fusion. More specifically, the proximal end face of the distal member 102 at the proximal end part 102a is joined to the distal end face of the catheter body 103 at the most distal end part 103b by fusion. The distal member 102 and the catheter body 103 share the central axis O, and the inner surfaces of the distal member 102 and the catheter body 103 are connected with substantially uniform inner diameters without a step.

The layer structure and materials of the distal member 102, the inner tube 108, and the balloon 109 may be the same as those described in conjunction with the previous embodiment above.

An axially intermediate part 102c, which is a section connecting the distal end part 102b and the proximal end part 102a in the distal member 102, has a bending portion 110 which bends (in other words, is curved) when an external force F (see, e.g., FIG. 4) in the bending direction is applied to the distal end part 102b of the distal member 102 with the distal end part 103a of the catheter body 103 being fixed. In this manner, the bending portion 110 of the distal member 102 is positioned distal to the most distal end part 103b of the catheter body 103 (that is, the most distal end part of the inner tube 108). The bending portion 110 is located distal to the proximal end part 102a of the distal member 102. The bending portion 110 is located distal to the most distal end part 109d of the balloon 109. The bending portion 110 of the distal member 102 is located proximal to the distal end part 102b of the distal member 102.

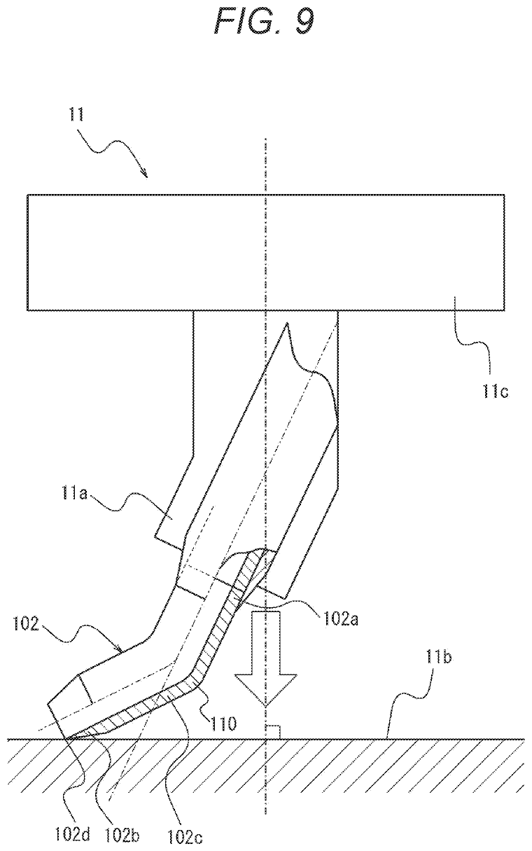

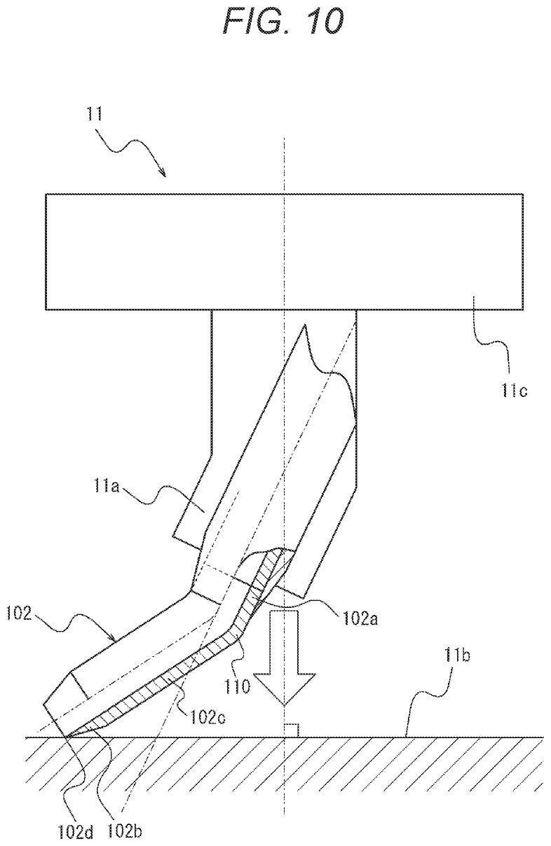

The position of the bending portion 110 can be confirmed, for example, by a bending test illustrated in FIGS. 9 and 10 as an example. The section that bends as illustrated in FIG. 9 by the test described with reference to FIG. 4 is the bending portion 110. Since the Young's modulus of the bending portion 110 is smaller than the Young's modulus of the proximal end part 102a of the distal member 102, the bending portion 110 is positioned distal to the most distal end part 103b of the catheter body 103. More specifically, the bending portion 110 is located distal to the proximal end part 102a in the vicinity of a region distal to the most distal end part 109d of the balloon 109. In contrast to the present embodiment, in a comparative example in which the Young's modulus of the distal member 102 is constant over the entire length in the axial direction, the bending portion 110 is located at the proximal end part 102a of the distal member 102, more specifically, at the proximal end part 102a in the vicinity of a region distal to the most distal end part 109d of the balloon 109, as illustrated in FIG. 10. Alternatively, the distal member 102 of the comparative example may buckle in a bellows shape over the entire length in the axial direction without forming the bending portion 110.

The bending portion 110 of the present embodiment can be formed through a heat treatment step of performing a heat treatment for fusing the proximal end part 102a of the distal member 102 to the catheter body 103 while adjusting a thermal load on the axially intermediate part 102c of the distal member 102 to be smaller than a thermal load on the proximal end part 102a of the distal member 102.

The heat treatment step includes a distal end treatment step of applying a thermal load larger than the thermal load to the axially intermediate part 102c of the distal member 102 to the distal end part 102b of the distal member 102. The distal end part 102b of the distal member 102 is formed in a tapered shape by the distal end treatment step. The distal end part 102b may be formed in a rounded shape by the distal end treatment step.

The heat treatment step also includes a heat transfer step of transferring heat to the proximal end part 102a of the distal member 102 via a cylindrical first heat transfer portion 12 that contracts by heat and transferring heat to the distal end part 102b of the distal member 102 via a cylindrical second heat transfer portion 13 that contracts by heat (see, e.g., FIG. 7). Due to the heat transfer step described above, a thermal load larger than the thermal load to the axially intermediate part 102c of the distal member 102 can be applied to the proximal end part 102a and the distal end part 102b of the distal member 102.

Due to the suppression of the thermal load on the axially intermediate part 102c of the distal member 102, it is possible to prevent a situation in which a thermoplastic resin constituting the axially intermediate part 102c of the distal member 102 is melted and cured again to cause a change in composition, so that the thermoplastic resin is harder than before being melted, that is, to prevent a decrease in flexibility (that is, an increase in Young's modulus) due to the thermal load on the axially intermediate part 102c of the distal member 102.

In the heat treatment step, heat transfer and a contraction force due to heat are applied to the distal end part 109b of the balloon via the first heat transfer portion 12. As a result, the distal end part 109b of the balloon is fused to the proximal end part 102a of the distal member 102 and the distal end part 103a of the catheter body 103. The materials of the distal end part 109b of the balloon, the proximal end part 102a, and the distal end part 103a are melted to form a melt-solidified body. The distal end part 109b of the balloon has a structure in which the thickness gradually decreases to the most distal end part 109d by the contraction force of the molten material due to the heat transfer of the first heat transfer portion 12.

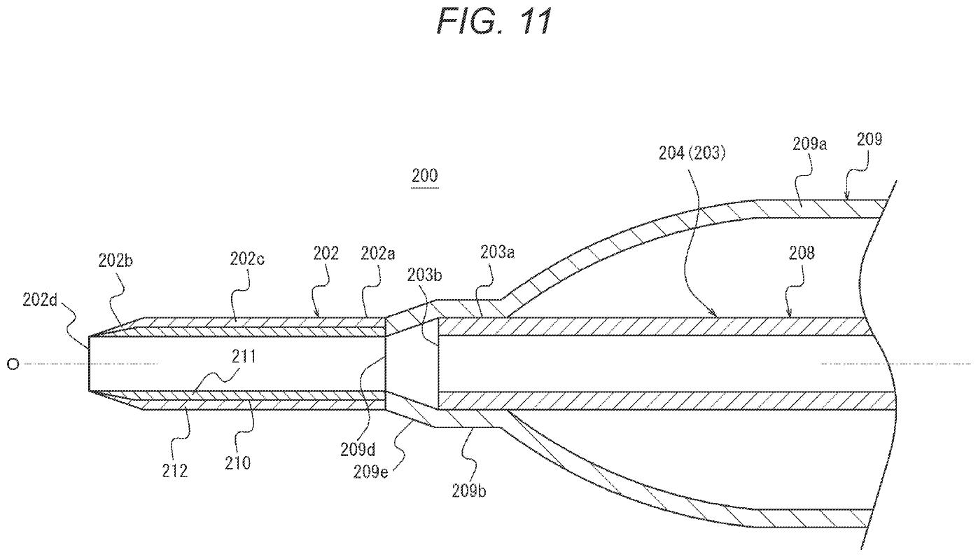

Next, a catheter 200 according to another embodiment of the present disclosure will be described with reference to FIG. 11, that is a partially enlarged view of the catheter 200. Note that the components of FIG. 11 that are common to FIGS. 1 and 2 are denoted by the same reference numerals as those in FIGS. 1 and 2, and the details thereof are assumed to be the same and are omitted.

As illustrated in FIG. 11, the catheter 200 according to another embodiment includes a cylindrical distal member 202 extending along a central axis O, and a catheter body 203 including a distal end part 203a located proximal to a proximal end part 202a of the distal member 202. The catheter body 203 includes a shaft portion 204 having the distal end part 203a separated from the proximal end part 202a of the distal member 202 with a gap and having an elongated shape coaxial with the distal member 202, and a hub 5 having a distal end part connected to a proximal end part of the shaft portion 204.

The balloon 209 has a cylindrical shape in which a distal end part 209b and a proximal end part extend in the axial direction, and an axially intermediate part between the distal end part and the proximal end part constitutes a cylindrical balloon body 209a enlarged in the radial direction. The distal end part 209b of the balloon 209 covers the distal end part of an inner tube 208 (the distal end part 203a of the catheter body 203). The distal end part 209b extends to the distal side beyond the most distal end part 203b as an inclined portion 209e. The inclined portion 209e decreases in diameter toward the distal end. The distal end face of the balloon 209 at the most distal end part 209d is joined to the proximal end face of the distal member 202 at the proximal end part 202a by fusion.

The distal member 202 has a two-layer structure of an inner layer 211 and an outer layer 212. The inner layer 211 is made of a material harder than the outer layer 212. A material that is less likely to soften even when the distal member 202 is inserted into the body and that suppresses a decrease in slidability of the guide wire is selectable. Such a material is, for example, high density polyethylene. The outer layer 212 is made of a material having more excellent flexibility than the inner layer 211. Thus, it is possible to suppress damage to a living body such as a blood vessel. Such a material is, for example, a polyamide-based elastomer. The distal member 202 may have a three-layer structure including an intermediate layer between the inner layer 211 and the outer layer 212. The distal member 202 may have a single layer. In a single-layer structure, a material used for the inner layer 211 can be adopted.

The layer structure and materials of the inner tube 208 and the balloon 209 may be the same as, or similar to, those in at least one of the embodiments previously described above.

An axially intermediate part 202c, which is a section connecting the distal end part 202b and the proximal end part 202a in the distal member 202, has a bending portion 210 which bends (in other words, is curved) when an external force F (see, e.g., FIG. 4) in the bending direction is applied to the distal end part 202b of the distal member 202 with the proximal end part 202a being fixed. In this manner, the bending portion 210 of the distal member 202 is positioned distal to the most distal end part 203b of the catheter body 203 (that is, the most distal end part of the inner tube 208). The bending portion 210 of the distal member 202 is located distal to the proximal end part 202a of the distal member 202. The bending portion 210 of the distal member 202 is located proximal to the distal end part 202b of the distal member 202.

The bending portion 210 of the present embodiment can be formed through a heat treatment step of performing a heat treatment for fusing the proximal end part 202a of the distal member 202 to the distal end part 209b of the balloon 209 while adjusting a thermal load on the axially intermediate part 202c of the distal member 202 to be smaller than a thermal load on the proximal end part 202a of the distal member 202.

The heat treatment step includes a distal end treatment step of applying a thermal load larger than the thermal load to the axially intermediate part 202c of the distal member 202 to the distal end part 202b of the distal member 202. The distal end part 202b of the distal member 202 is formed in a tapered shape by the distal end treatment step. The distal end part 202b may be formed in a rounded shape by the distal end treatment step.

The heat treatment step also includes a heat transfer step of transferring heat to the proximal end part 202a of the distal member 202 via a cylindrical first heat transfer portion 12 that contracts by heat and transferring heat to the distal end part 202b of the distal member 202 via a cylindrical second heat transfer portion 13 that contracts by heat (see, e.g., FIG. 7). Due to the heat transfer step described above, a thermal load larger than the thermal load to the axially intermediate part 202c of the distal member 202 can be applied to the proximal end part 202a and the distal end part 202b of the distal member 202.

In the heat treatment step, heat transfer and a contraction force due to heat are applied to the distal end part 209b of the balloon via the first heat transfer portion 12. As a result, the distal end part 209b of the balloon is fused to the distal end part 203a of the catheter body 203. The materials of the distal end part 209b of the balloon and the distal end part 203a are melted to form a melt-solidified body.

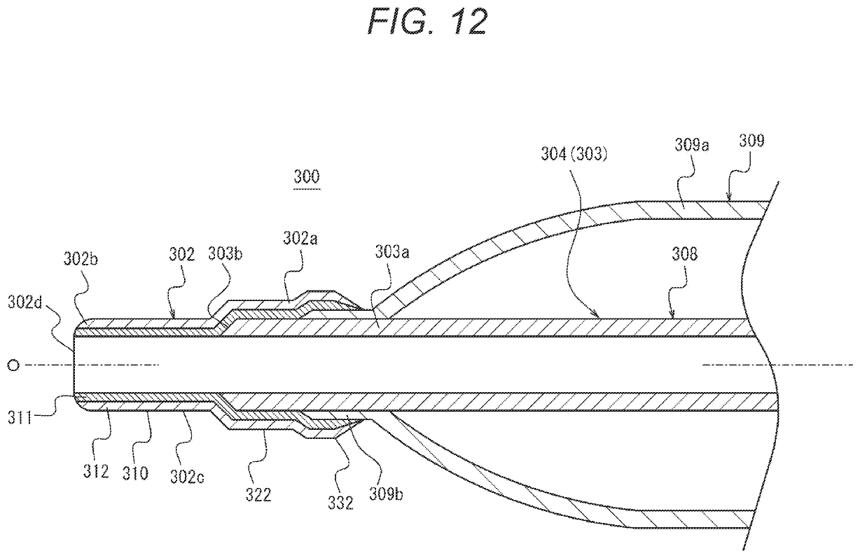

Next, a catheter 300 according to another embodiment will be described with reference to FIG. 12, that is a partially enlarged view of the catheter 300. Note that the components of FIG. 12 that are common to FIGS. 1 and 2 are denoted by the same reference numerals as those in FIGS. 1 and 2, and the details thereof are assumed to be the same and are omitted.

As illustrated in FIG. 12, the catheter 300 according to the other embodiment includes a cylindrical distal member 302 extending along a central axis O, and a catheter body 303 including a distal end part 303a located between a proximal end part 302a and a distal end part 302b of the distal member 302. The distal member 302 includes a catheter body covering portion 322, which covers and fixes the distal end part 303a of the catheter body 303 by fusion, and a balloon distal-end covering portion 332, which is connected to the catheter body covering portion 322 and covers and fixes at least a part of a distal end part 309b of a balloon 309 by fusion. The outer diameter of the balloon distal-end covering portion 332 is larger than the outer diameter of the catheter body covering portion 322. The balloon distal-end covering portion 332 is connected to the catheter body covering portion 322 with a tapered transition portion therebetween.

The balloon 309 has a cylindrical shape in which the distal end part 309b and the proximal end part extend in the axial direction, and an axially intermediate part between the distal end part and the proximal end part constitutes a cylindrical balloon body 309a enlarged in the radial direction. The distal end part 309b of the balloon 309 is joined to the distal end part of an inner tube 308 (the distal end part 303a of the catheter body 303) by fusion. The balloon 309 is disposed such that at least a part of the distal end part 309b of the balloon 309 is sandwiched between the proximal end part 302a of the distal member 302 and the catheter body 303.

The distal member 302 has a two-layer structure of an inner layer 311 and an outer layer 312. The materials described in the previous embodiment can be used as a material of each layer. As the inner layer 311, a material having high compatibility with a material constituting the outer surface of the catheter body 303 and a material constituting the inner surface of the distal end part 309b of the balloon 309 can be suitably selected. The distal member 302 may have a three-layer structure including an intermediate layer between the inner layer 311 and the outer layer 312. The distal member 302 may have a single layer. In a single-layer structure, a material used for the inner layer 311 can be adopted.

An axially intermediate part 302c, which is a section connecting the distal end part 302b and the most distal end part 303b of the catheter body 303 in the distal member 302, has a bending portion 310 which bends (in other words, is curved) when an external force F (see, e.g., FIG. 4) in the bending direction is applied to the distal end part 302b of the distal member 302 with the distal end part 303a of the catheter body 303 being fixed. In this manner, the bending portion 310 of the distal member 302 is positioned distal to the most distal end part 303b of the catheter body 303 (that is, the most distal end part of the inner tube 308). The bending portion 310 of the distal member 302 is located proximal to the distal end part 302b of the distal member 302.

The bending portion 310 of the present embodiment can be formed through a heat treatment step of performing a heat treatment for fusing the proximal end part 302a of the distal member 302 to the distal end part 303a of the catheter body 303 and the distal end part 309b of the balloon 309 while adjusting a thermal load on the axially intermediate part 302c of the distal member 302 to be smaller than a thermal load on the proximal end part 302a of the distal member 302.

The heat treatment step includes a distal end treatment step of applying a thermal load larger than the thermal load to the axially intermediate part 302c of the distal member 302 to the distal end part 302b of the distal member 302. The distal end part 302b of the distal member 302 may be formed in a rounded shape by the distal end treatment step. The distal end part 302b may be formed in a tapered shape by the distal end treatment step.

The heat treatment step also includes a heat transfer step of transferring heat to the proximal end part 302a of the distal member 302 via a cylindrical first heat transfer portion 12 that contracts by heat and transferring heat to the distal end part 302b of the distal member 302 via a cylindrical second heat transfer portion 13 that contracts by heat (see, e.g., FIG. 7). Due to the heat transfer step described above, a thermal load larger than the thermal load to the axially intermediate part 302c of the distal member 302 can be applied to the proximal end part 302a and the distal end part 302b of the distal member 302.

In the heat treatment step, heat transfer and a contraction force due to heat are applied to the proximal end part 302a of the distal member 302 via the first heat transfer portion 12. As a result, the proximal end part 302a of the distal member 302 is individually fused to the distal end part 309b of the balloon and the distal end part 303a of the catheter body.

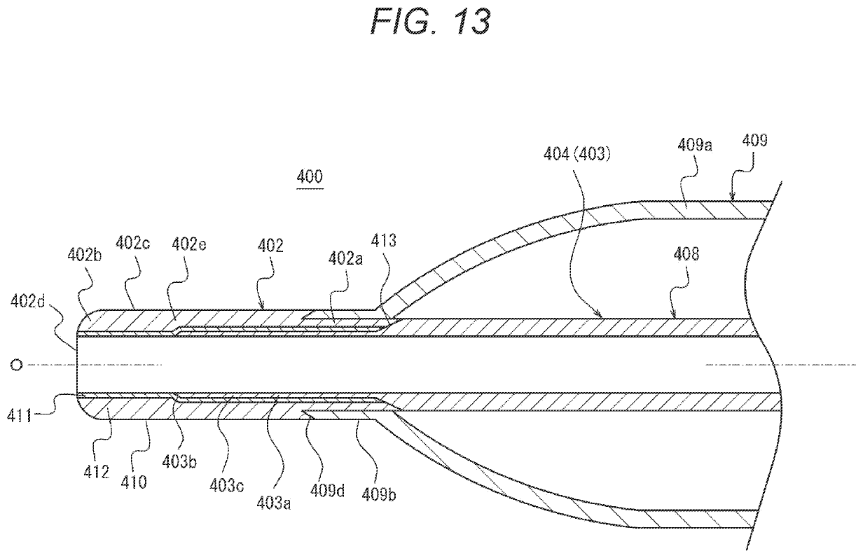

Next, a catheter 400 according to another embodiment will be described with reference to FIG. 13, that is a partially enlarged view of the catheter 400. Note that the components of FIG. 13 that are common to FIGS. 1 and 2 are denoted by the same reference numerals as those in FIGS. 1 and 2, and the details thereof are assumed to be the same and are omitted.

As illustrated in FIG. 13, the catheter 400 according to the other embodiment includes a cylindrical distal member 402 extending along a central axis O, and a catheter body 403 including a most distal end part 403b located between a proximal end part 402a and a distal end part 402b of the distal member 402. The distal end part 403a of the catheter body 403 has a portion having a narrow outer diameter at a step 413, and terminates at the most distal end part 403b via a small diameter portion 403c that keeps the narrow outer diameter in the distal direction. The inner surface of the distal member 402 is covered with and fused to the outer surface of the small diameter portion 403c.

The balloon 409 has a cylindrical shape in which the distal end part 409b and the proximal end part extend in the axial direction, and an axially intermediate part between the distal end part and the proximal end part constitutes a cylindrical balloon body 409a enlarged in the radial direction. The distal end part 409b of the balloon 409 is fused to and covers at least a part of the proximal end part 402a of the distal member 402. The distal end part 409b of the balloon 409 and the distal end part 403a of the catheter body 403 are disposed so as to sandwich the proximal end part 402a of the distal member 402. The outer diameter of the distal end part 409b of the balloon 409 is smoothly connected to the outer diameter of the distal member 402 without a step.

The distal member 402 has a two-layer structure of an inner layer 411 and an outer layer 412. The materials described in the previous embodiment can be used as a material of each layer. As the inner layer 411, a material having high compatibility with a material constituting the outer surface of the catheter body 403 can be suitably selected. As the outer layer 412, a material having high compatibility with a material constituting the inner surface of the distal end part 409b of the balloon 409 can be suitably selected. The distal member 402 may have a three-layer structure including an intermediate layer between the inner layer 411 and the outer layer 412. The distal member 402 may have a single layer. In a single-layer structure, a material used for the inner layer 411 can be adopted.

An axially intermediate part 402c, which is a section connecting the distal end part 402b and the most distal end part 403b of the catheter body 403 in the distal member 402, has a bending portion 410 which bends (in other words, is curved) when an external force F (see, e.g., FIG. 4) in the bending direction is applied to the distal end part 402b of the distal member 402 with the distal end part 403a of the catheter body 403 being fixed. In this manner, the bending portion 410 of the distal member 402 is positioned distal to the most distal end part 403b of the catheter body 403 (that is, the most distal end part of an inner tube 408). The bending portion 410 is positioned distal to a section 402e of the distal member 402 in the vicinity of a region distal to the most distal end part 403b of the catheter body 403 (that is, the most distal end part of the inner tube 408). The bending portion 410 of the distal member 402 is located proximal to the distal end part 402b of the distal member 402.

The bending portion 410 of the present embodiment can be formed through a heat treatment step of performing a heat treatment for fusing the proximal end part 402a of the distal member 402 to the distal end part 403a of the catheter body 403 and for fusing the distal end part 409b of the balloon 409 to the proximal end part 402a of the distal member 402 while adjusting a thermal load on the axially intermediate part 402c of the distal member 402 to be smaller than a thermal load on the proximal end part 402a of the distal member 402.

The heat treatment step includes a distal end treatment step of applying a thermal load larger than the thermal load to the axially intermediate part 402c of the distal member 402 to the distal end part 402b of the distal member 402. The distal end part 402b of the distal member 402 may be formed in a rounded shape by the distal end treatment step. The distal end part 402b may be formed in a tapered shape by the distal end treatment step.

The heat treatment step also includes a heat transfer step of transferring heat to the distal member 402 covering the small diameter portion 403c of the catheter body 403 and to the distal end part 409b of the balloon 409 via a cylindrical first heat transfer portion 12 that contracts by heat and transferring heat to the distal end part 402b of the distal member 402 via a cylindrical second heat transfer portion 13 that contracts by heat (see, e.g., FIG. 7). Due to the heat transfer step described above, a thermal load larger than the thermal load to the axially intermediate part 402c of the distal member 402 can be applied to the distal member 402 covering the distal end part 403a of the catheter body 403 and to the distal end part 402b.

In the heat treatment step, heat transfer and a contraction force due to heat are applied to the distal member 402 covering the distal end part 403a of the catheter body 403 and to the distal end part 409b of the balloon 409 via the first heat transfer portion 12. The distal member 402 covering the distal end part 403a of the catheter body 403 is fused to the distal end part 403a of the catheter body 403. The distal end part 409b of the balloon is fused to the proximal end part 402a of the distal member 402.

The exemplary systems and methods of this disclosure have been described in relation to a catheter and method for manufacturing the same. However, to avoid unnecessarily obscuring the present disclosure, the preceding description omits a number of known structures and devices. This omission is not to be construed as a limitation of the scope of the claimed disclosure. Specific details are set forth to provide an understanding of the present disclosure. It should, however, be appreciated that the present disclosure may be practiced in a variety of ways beyond the specific detail set forth herein.

A number of variations and modifications of the disclosure can be used. It would be possible to provide for some features of the disclosure without providing others.

References in the specification to “one embodiment,” “an embodiment,” “an example embodiment,” “some embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in conjunction with one embodiment, it is submitted that the description of such feature, structure, or characteristic may apply to any other embodiment unless so stated and/or except as will be readily apparent to one skilled in the art from the description. The present disclosure, in various embodiments, configurations, and aspects, includes components, methods, processes, systems and/or apparatus substantially as depicted and described herein, including various embodiments, subcombinations, and subsets thereof. Those of skill in the art will understand how to make and use the systems and methods disclosed herein after understanding the present disclosure. The present disclosure, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the disclosure.

Moreover, though the description of the disclosure has included description of one or more embodiments, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and this disclosure.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

What is claimed is:

1. A catheter comprising:
a distal member having a cylindrical shape and connected to a distal end part of a catheter body, wherein
the distal member has a bending portion located distal to a most distal end part of the catheter body, the bending portion being a section that bends when an external force in a bending direction is applied to a distal end part of the distal member with the distal end part of the catheter body being fixed.

2. The catheter according to claim 1, wherein the distal end part of the distal member is tapered.

3. The catheter according to claim 1, wherein the distal member is formed of a thermoplastic resin.

4. The catheter according to claim 3, wherein the distal member includes only at least one thermoplastic resin layer.

5. The catheter according to claim 4, wherein the bending portion has a Young's modulus smaller than a Young's modulus of a proximal end part of the distal member.

6. The catheter according to claim 1, wherein the distal member comprises a proximal end part disposed opposite the distal end part of the distal member, wherein the bending portion is arranged between the proximal end part of the distal member and the distal end part of the distal member, and wherein the distal member is connected to the distal end part of the catheter body at the proximal end part of the distal member.

7. The catheter according to claim 6, wherein the proximal end part of the distal member, the bending portion, and the distal end part of the distal member are made from a single unitary piece of material.

8. The catheter according to claim 7, wherein the proximal end part of the distal member comprises a heat-transformed material section of the single unitary piece of material having a greater Young's modulus than a Young's modulus of the bending portion.

9. The catheter according to claim 7, wherein the distal end part of the distal member comprises a heat-transformed material section of the single unitary piece of material having a greater Young's modulus than a Young's modulus of the bending portion.

10. A catheter comprising:
a distal member having a cylindrical shape and connected to a distal end part of a catheter body, wherein
the distal member has a bending portion having a Young's modulus smaller than a Young's modulus of a proximal end part of the distal member, the bending portion being a section that bends when an external force in a bending direction is applied to a distal end part of the distal member with the distal end part of the catheter body being fixed.

11. The catheter according to claim 10, wherein the Young's modulus of the bending portion of the distal member is smaller than a Young's modulus of the distal end part of the distal member.

12. The catheter according to claim 11, wherein the bending portion is arranged between the proximal end part of the distal member and the distal end part of the distal member, and wherein the distal member is connected to the distal end part of the catheter body at the proximal end part of the distal member.

13. The catheter according to claim 12, wherein the proximal end part of the distal member, the bending portion, and the distal end part of the distal member are made from a single unitary piece of material.

14. The catheter according to claim 13, wherein the proximal end part of the distal member comprises a melted-and-cured material section of the single unitary piece of material.

15. The catheter according to claim 14, wherein the Young's modulus of the proximal end part of the distal member is equal to the Young's modulus of the distal end part of the distal member.

16. A method for manufacturing a catheter, the method comprising:
a heat treatment step of performing a heat treatment for fusing a proximal end part of a distal member having a cylindrical shape to a catheter body while adjusting a thermal load on an axially intermediate part of the distal member to be smaller than a thermal load on the proximal end part.

17. The method according to claim 16, wherein the heat treatment step includes a heat transfer step of transferring heat to the proximal end part through a heat transfer portion that has a cylindrical shape and that contracts by heat.

18. The method according to claim 17, wherein the heat transfer portion absorbs radiation and generates heat.

19. The method according to claim 16, wherein the heat treatment step includes a distal end treatment step of applying a thermal load larger than a thermal load on the axially intermediate part of the distal member to a distal end part of the distal member.

20. The method according to claim 16, wherein the heat treatment step further comprises:
arranging the distal member inside a tubular heat treatment member the tubular heat treatment member comprising a first heat transfer portion arranged at a first end of the tubular heat treatment member and a second heat transfer portion arranged at a second end of the tubular heat treatment member, wherein the first heat transfer portion is joined to the second heat transfer portion by a connection portion, wherein the first heat transfer portion and the second heat transfer portion comprise a colored tube that absorbs laser energy radiation, and wherein the connection portion comprises a transparent tube that prevents absorbing laser energy radiation; and
applying a laser radiation to the first heat transfer portion and the second heat transfer portion causing the first heat transfer portion to melt and cure the proximal end part of the distal member into a heat-transformed state having a Young's modulus greater than a Young's modulus of the axially intermediate part of the distal member.
